(19) United States
(12) Patent Application Publication
(10) Pub. No.: US 2016/0184442 A1
(43) Pub. Date: Jun. 30, 2016

(54) PHARMACEUTICAL COMPOSITIONS FOR TRANSDERMAL DELIVERY OF ACTIVE AGENTS

(71) Applicants: Mark Linford, Bendigo (AU); Jeremy Delk, Nicholasville, KY (US)

(72) Inventors: Mark Linford, Bendigo (AU); Jeremy Delk, Nicholasville, KY (US)

(21) Appl. No.: 14/852,017

(22) Filed: Sep. 11, 2015

Related U.S. Application Data
(60) Provisional application No. 62/049,035, filed on Sep. 11, 2014.

(51) Int. Cl. A61K 47/44; A61K 47/10; A61K 31/465; A61K 39/00; A61K 38/00

(52) U.S. Cl. CPC A61K 47/44; A61K 39/00; A61K 38/00; A61K 31/465; A61K 47/10

(57) ABSTRACT

The present invention relates to novel compositions that are effective for delivering active pharmaceutical agents transdermally. The compositions comprise a mixture of water, propylene glycol, phenoxyethanol, heavy paraffin oil, soft white paraffin, cetostearyl alcohol, and macrogol cetostearyl ether and demonstrate improved delivery of active agents when formulated together.

### RELATED APPLICATIONS

This application claims priority to U.S. Provisional Application 62/049,038, filed Sep. 11, 2014, the contents of which are hereby incorporated in its entirety. This application is also related to U.S. Provisional Patent Application Ser. No. 61/989,042, filed May 6, 2014, and U.S. patent application Ser. No. 14/705,393, filed May 6, 2015, both of which are also incorporated by reference it their entirety.

### FIELD OF THE INVENTION

The present invention provides for novel pharmaceutical carriers and compositions for improved transdermal delivery of active agents.

### BACKGROUND

Topical application of pharmaceutical compositions is often ineffective in administering a drug, as the physiological make-up of the skin makes this organ difficult to penetrate. However, applications such site specific delivery to the epidermis, dermis and tissues beneath, as well as avoiding metabolic organs such as the liver and kidneys that can chemically alter an active agent following systemic delivery, make overcoming the barrier desirable.

An often used approach to deliver an agent across the skin is through disrupting the barrier through physical and chemical means. This can result in pain as well as vulnerability to infection in the subject. Accordingly, a non-disruptive agent to assist in carrying an active agent into and/or across the epidermis and dermis is attractive. The present invention provides a composition of a pharmaceutical carrier that transdermally delivers active agents.

### SUMMARY OF THE INVENTION

The present invention provides a pharmaceutically acceptable carrier comprising water, propylene glycol, phenoxyethanol, heavy paraffin oil, soft white paraffin, cetostearyl alcohol and macrogol cetrostearyl ether. The present invention further provides for formulations comprising the carrier described herein and an active agent, such as a pharmaceutical compound.

In one embodiment, the pharmaceutically acceptable carrier includes between about 46.5 and about 49.5 weight percent water, about 4.2 and about 5.8 weight percent propylene glycol, about 0.4 and about 0.8 weight percent phenoxyethanol, about 9.2 and about 10.8 weight percent heavy paraffin oil, about 9.2 and about 10.8 weight percent soft white paraffin, about 14.2 and about 15.6 weight percent cetostearyl alcohol, and about 0.4 and about 1.0 weight percent macrogol cetostearyl ether.

In one embodiment, the pharmaceutically acceptable carrier includes about 47.9 weight percent water, 5.0 weight percent propylene glycol, 0.6 weight percent phenoxyethanol, 10.0 weight percent heavy paraffin oil, 10.0 weight percent soft white paraffin, 14.8 weight percent cetostearyl alcohol and 0.7 weight percent macrogol cetostearyl ether.

### BRIEF DESCRIPTION OF THE DRAWINGS

FIG. 1 shows that tumor treatment with tea tree oil and the carrier does not cause dermal inflammation by skin inflammation score.

FIG. 2 shows that inflammatory cell infiltration is similar to untreated skin as compared to treated with DMSO.

FIG. 3 shows that the applied tea tree oil with the carrier inhibits B16 melanoma growth in the murine model.

FIG. 4 shows a comparison of the subsequent tumor weights in treated and untreated subjects.

FIG. 5 shows three independent studies of the effect of tea tree oil and the carrier on tumor volume.

FIG. 6 shows a comparison between treatment and non-treatment applied on day three after tumor implantation.

FIG. 7 shows a comparison of subcutaneous tumor size between treated and non-treated subjects.

FIG. 8 shows mean tumor weight between treated and untreated subjects.

### DETAILED DESCRIPTION

The present invention provides for a novel composition that has demonstrated efficacy as a carrier for transdermal delivery of active agents. The carrier may comprise water, propylene glycol, phenoxyethanol, heavy paraffin oil, soft white paraffin, cetostearyl alcohol, and macrogol cetostearyl ether. The carrier may comprise between about 45 to 50 weight percent water, about 4 to 6 weight percent propylene glycol, about 0.2 to 1 weight percent phenoxyethanol, about 5 to 15 weight percent heavy paraffin oil, about 5 to 15 weight percent soft white paraffin, about 10 to 20 weight percent cetostearyl alcohol, and about 0.2 to 2 weight percent macrogol cetostearyl ether.

The present invention provides for a pharmaceutical composition of an active agent and the carrier of the present invention. In one embodiment, the pharmaceutical composition is a topical cream including a pharmaceutically acceptable carrier (such as the carrier disclosed in co-pending U.S. Provisional Patent Application Ser. No. 61/989,042, incorporated herein by reference) and an active agent.

In one embodiment, the pharmaceutical composition includes about 86 to about 90 weight percent of the pharmaceutically acceptable carrier.

In one embodiment, the pharmaceutical composition includes about 5 to about 20 percent active agent. In one embodiment, the pharmaceutical composition includes about 7 to 15 percent active agent. In one embodiment, the pharmaceutical composition includes about 9 to 14 percent active agent. In one embodiment, the pharmaceutical composition includes about 9 to 10 percent active agent.

Those skilled in the art will appreciate that active agents comprise pharmaceutical agents known in the art (see, e.g., Physicians' Desk Reference (65th ed., 2010). Montvale, N.J.: PDR Network; www.drugs.com/drug_information.html; www.rxlist.com). Those skilled in the art will also appreciate that the carrier composition described herein can be combined with other known carriers in the art (see, e.g., Remington: The Science and Practice of Pharmacy. 21st Edition. Philadelphia, Pa. Lippincott Williams & Wilkins. 2005). Those skilled in the art will appreciate that the therapeutic agent can be any known therapeutic, such as a hormone, small molecule, peptide, nucleotide, vitamin, vaccine, antigen, antibody or fragment thereof, or organic chemical. Those skilled in the art will appreciate that the therapeutic agent can be applied with the carrier to a subject at any point of a subject's skin. Those skilled in the art will appreciate that the composition can be applied at a particular site, such as a site in need of treatment. Those skilled in the art will further appreciate that application of the composition at any point can further provide systemic administration, such that the agent can be delivered to a site in need thereof that is distal to the point of application. Those skilled in the art will further appreciate that the therapeutic agent can comprise more than one agent. For example, the therapeutic agent may comprise an anti-cancer agent, such as a skin cancer agent (e.g. tea tree oil, fluorouracil, imiquimod, visodegib, aldesleukin, dabrafenib, dacarbazine, recombinant interferon α-2b, ipilimumab, pembrolizumab, trametinib, nivulomab, and vemurafenib).

In one embodiment, the pharmaceutically acceptable carrier includes water, propylene glycol, phenoxyethanol, heavy paraffin oil, soft white paraffin, cetostearyl alcohol and macrogol cetrostearyl ether.

In one embodiment, the pharmaceutically acceptable carrier includes between about 46.5 and about 49.5 weight percent water, about 4.2 and about 5.8 weight percent propylene glycol, about 0.4 and about 0.8 weight percent phenoxyethanol, about 9.2 and about 10.8 weight percent heavy paraffin oil, about 9.2 and about 10.8 weight percent soft white paraffin, about 14.2 and about 15.6 weight percent cetostearyl alcohol, and about 0.4 and about 1.0 weight percent macrogol cetostearyl ether. The carrier may comprise about 47.9 weight percent water, 5.0 weight percent propylene glycol, 0.6 weight percent phenoxyethanol, 10.0 weight percent heavy paraffin oil, 10.0 weight percent soft white paraffin, 14.8 weight percent cetostearyl alcohol and 0.7 weight percent macrogol cetostearyl ether.

In one embodiment, the pharmaceutically acceptable carrier includes about 47.9 weight percent water, 5.0 weight percent propylene glycol, 0.6 weight percent phenoxyethanol, 10.0 weight percent heavy paraffin oil, 10.0 weight percent soft white paraffin, 14.8 weight percent cetostearyl alcohol and 0.7 weight percent macrogol cetostearyl ether.

In one embodiment, the pharmaceutical active ingredient comprises a protein-based vaccine, such as an influenza vaccine, tetanus toxoid vaccine, anthrax protective antigen, HIV vaccine, measles vaccine, and hepatitis viral antigens.

In one embodiment, the pharmaceutical active ingredient comprises a nucleic-acid (RNA and DNA) based vaccine, including plasmids, viral vectors, and/or oligonucleotides.

In one embodiment, the pharmaceutical active ingredient comprises recombinant proteins and peptides, including recombinant hormones, recombinant growth factors, recombinant cytokines, recombinant colony stimulating factors, recombinant anticoagulants, recombinant interferons, recombinant thrombolytics, and/or recombinant antibody-based drugs.

In one embodiment, the pharmaceutical active ingredient comprises vaccine adjuvants, including alum, incomplete Freund's adjuvant, natural and synthetic pattern recognition ligands, and natural and/or synthetic bacterial cell wall components.

In one embodiment, the pharmaceutical active ingredient comprises natural and synthetic opioids, including burprenorphine, fentanyl, naloxone and/or other opioid agonists and antagonists.

In one embodiment, the pharmaceutical active ingredient comprises non-opioid analgesics and non-steroidal anti-inflammatory drugs, including indomethacin, piroxicam, and/or diclofenac.

In one embodiment, the pharmaceutical active ingredient comprises natural and synthetic protein and amino acid hormones, including insulin, growth hormone, prolactin, and/or epinephrine.

In one embodiment, the pharmaceutical active ingredient comprises natural and synthetic steroid hormones, including progesterones, estrogens, androgens, tamoxifen, and/or other steroid agonists and antagonists.

In one embodiment, the pharmaceutical active ingredient comprises nicotine and related alkaloids and derivatives.

In one possible embodiment, the pharmaceutical composition includes between about 5 and 15 weight percent pharmaceutical agent and 85 to 95 weight percent of the carrier described herein. The resulting formulation may comprise between about 9.2 and about 10.8 weight percent of an active agent and between about 87.95 and about 90.05 weight percent of the carrier described herein. That formulation may further comprise between about 0.75 and about 1.25 weight percent vanillin.

A method for transdermal delivery of an active agent comprises topically applying to a subject in need thereof a topical cream or pharmaceutical composition as hereto described.

A method of treating AK comprises topically applying to a subject in need a pharmaceutically effective amount of the pharmaceutical composition. Between about 0.5 and about 1 gram of the pharmaceutical composition is applied per 10 cm² of skin surface area between 2 and 3 times a day.

The pharmaceutical composition is prepared by combining/mixing appropriate amounts of water, propylene glycol and phenoxyethanol together and heating to between about 70-75° C. Appropriate amounts of heavy paraffin oil, soft white paraffin, cetostearyl alcohol and macrogel cetrostearyl ether are blended together in another vessel and heated to between about 75-80° C. All of the ingredients are then added together and mixed for about one minute or until homogenized.

The homogenized mixture is then cooled to about 40-45° C. and an appropriate amount of vanillin is then added with further mixing.

Afterwards, the mixture is allowed to stand for 48 hours at room temperature. An appropriate amount of tea tree oil is then added with continual mixing to homogenize the pharmaceutical composition.

The present invention further provides methods for administering a formulation comprising the carrier and an active agent to the skin a subject in need thereof. The formulation may comprise between 5-75 weight percent of the active agent and between 25-95 weight percent of the carrier. Those skilled in the art will appreciate that the formulation may comprise more than one active agent. Those skilled in the art will further appreciate that a second carrier may be introduced to further augment delivery. The formulation may be applied topically to the skin a subject, such as a human or other mammal, such as a cat, dog, pig, horse, mouse or other domesticated or livestock animal. The formulation may be applied to native skin or to skin prepared by washing and/or shaving the treatment area. The skin may be further pretreated, such as through application of a disinfectant.

The formulation may be applied to the skin of the subject in an amount necessary to deliver the dose required of the active agent. Those skilled in the art will appreciate that the formulation may be adjusted to allow for the desired dose to be administered. Following application of the formulation, the skin may be left exposed or wrapped or covered in a dressing to protect the site of application. The formulation may be applied in a series of administrations. Such regimens will be determined by the condition to be treated, as well as by the dose needed and the subject's own response to the applied formulation.

### Examples

Tea tree oil (TTO) can be effective in treating precancerous actinic keratosis (see, e.g., Demelza et al., Journal of Dermatological Science, Volume 67, Issue 2, August 2012, Pages 120-129). This study demonstrated that Tea Tree Oil (TTO) can significantly reduce the viability in vitro of 2 murine tumor cell lines: AE17 and B16 in a dose and time dependent manner. The in vivo part of the study showed that 3% and 10% TTO can inhibit tumor growth in mice. 10% TTO in DMSO was able to cause regression of tumors. DMSO can enhance penetration of substances through skin (transdermal enchancer). Unfortunately treatment with 10% TTO/DMSO was limited to 4 days limited to 4 days due to development of severe irritation, allowing regrowth of the tumors. The following example demonstrates that the carrier of the invention can deliver TTO without the irritation seen in other carriers such as DMSO.

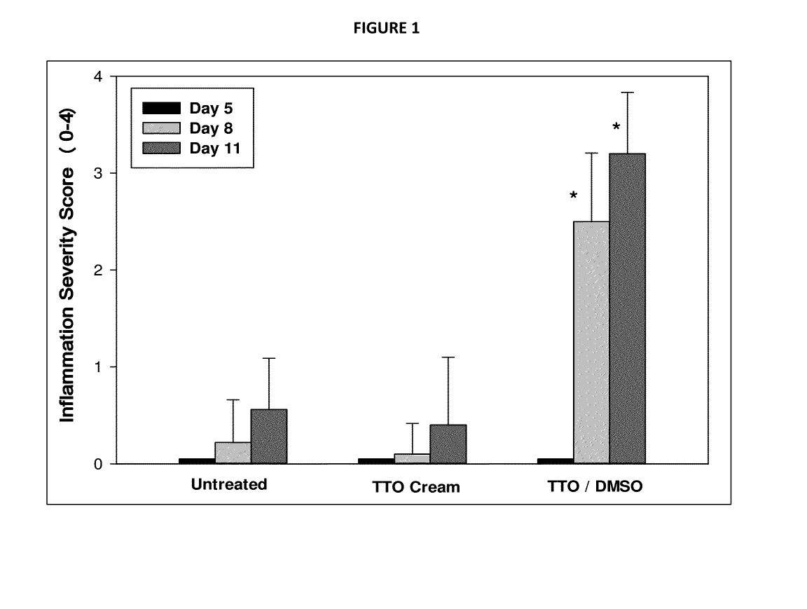

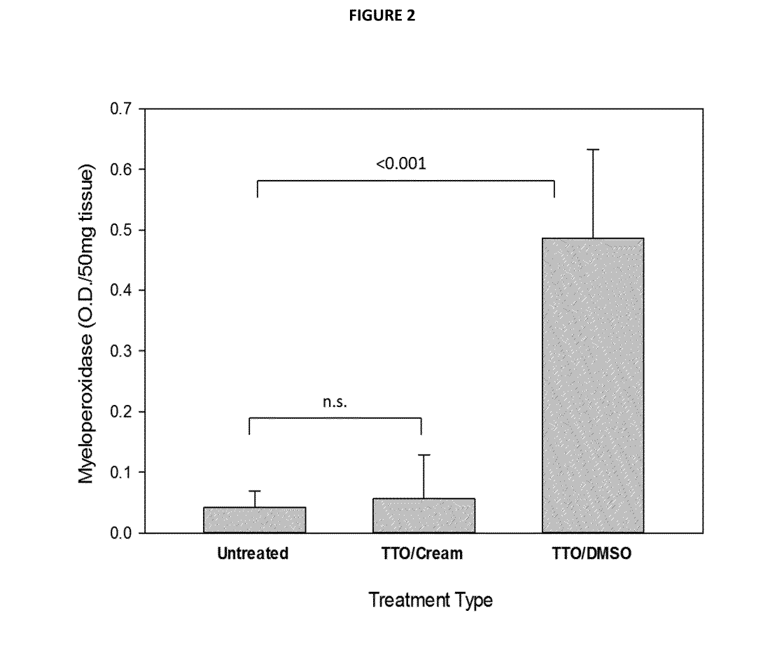

To test the carrier, tumors were implanted into murine models and allowed to grow for before treatment was implemented. Tea tree oil was mixed with the carrier and applied. FIG. 1 shows that application of the carrier did not result in an inflammatory response in the subjects. Further analysis of the presence of myeloperoxidase as a marker for inflammatory cell activity was performed. FIG. 2 shows that tea tree oil with DMSO resulted in the previously observed response, while tea tree oil with the carrier did not. These data confirm that both the carrier and tea tree oil do not trigger inflammation.

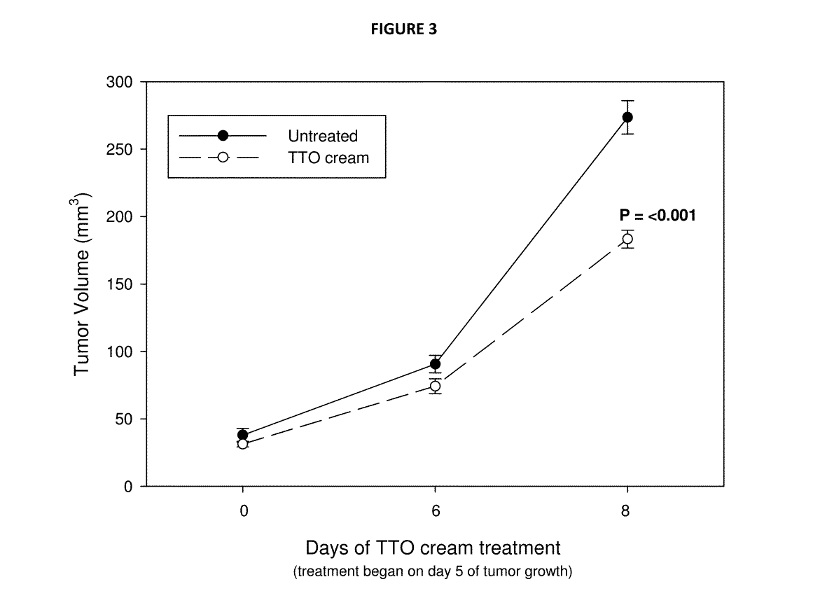

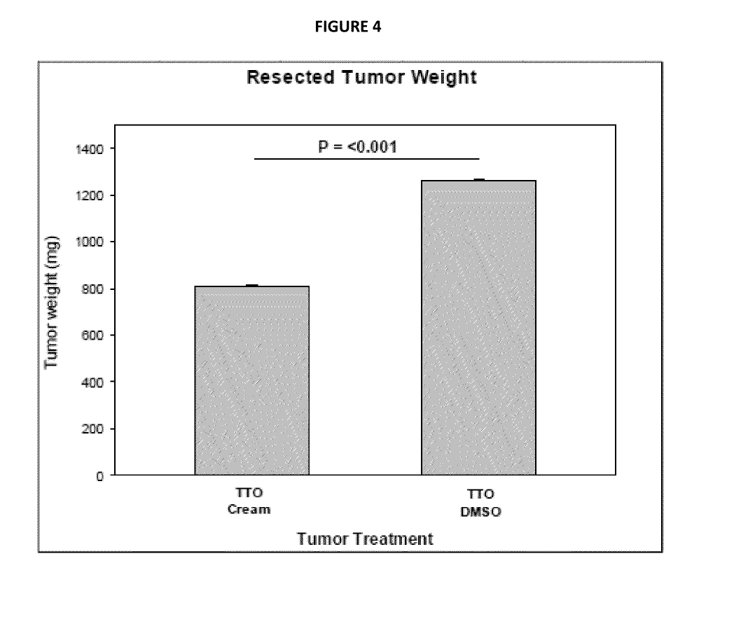

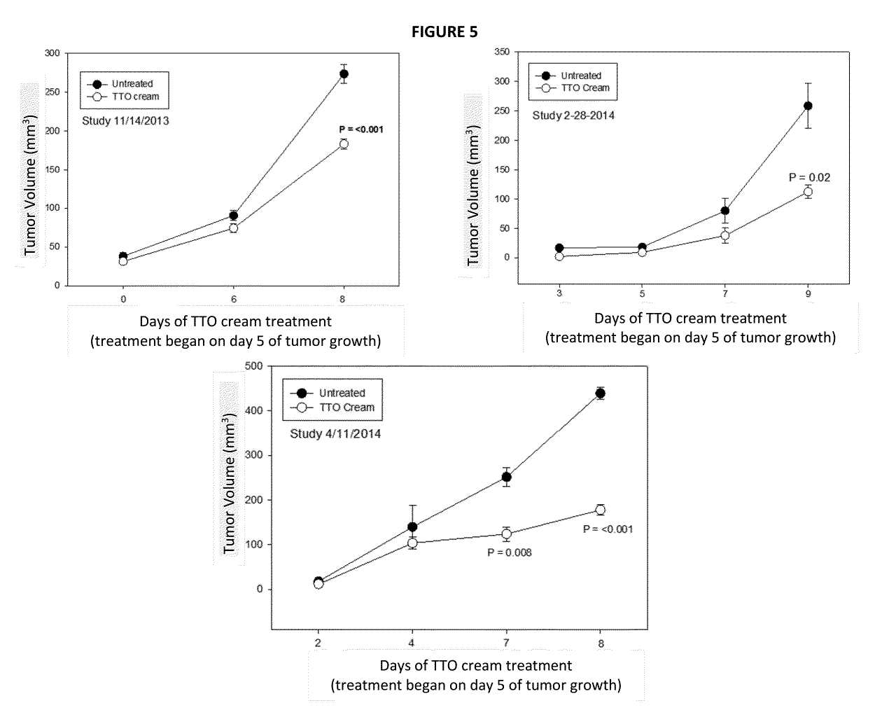

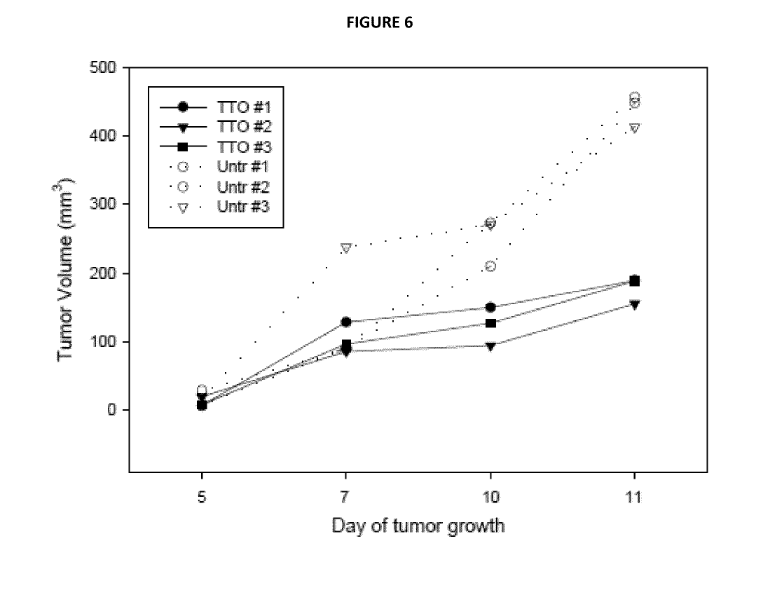

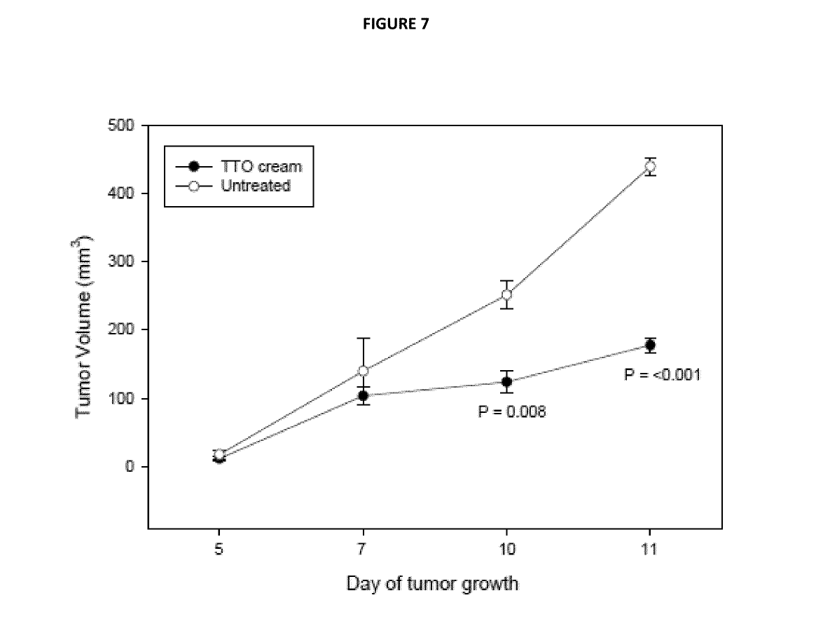

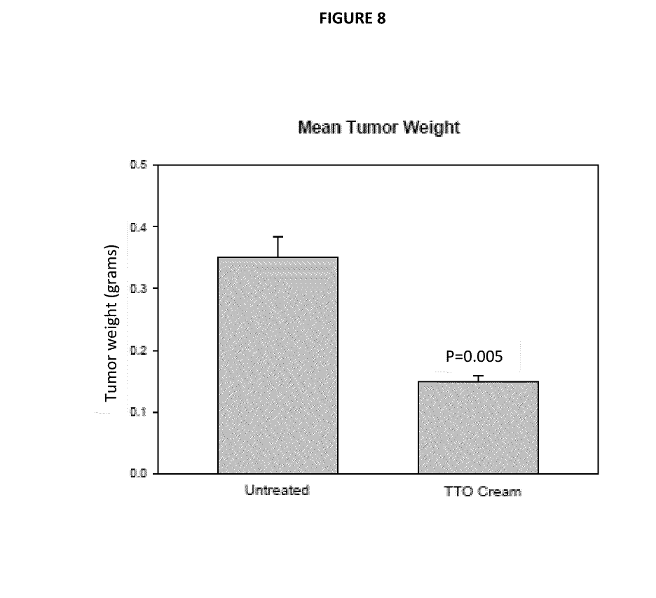

The effect of the TTO with the carrier on the size and weight of the tumors was also assessed. FIGS. 3-8 demonstrate that application of the tea tree oil with the carrier inhibited tumor progression. Collectively, the combination of the tea tree oil and the carrier were effective in delivering the desired therapeutic effect without triggering the undesired inflammatory response.

The foregoing descriptions of various embodiments provide illustration of the inventive concepts. The descriptions are not intended to be exhaustive or to limit the disclosed invention to the precise form disclosed. Modifications or variations are also possible in light of the above teachings. The embodiments described above were chosen to provide the best application to thereby enable one of ordinary skill in the art to utilize the inventions in various embodiments and with various modifications as are suited to the particular use contemplated. All such modifications and variations are within the scope of the invention. All publications, patents and patent applications referenced herein are to be each individually considered to be incorporated by reference in their entirety.

What is claimed:

1. A pharmaceutical composition, comprising:
a pharmaceutically acceptable carrier comprising water, propylene glycol, phenoxyethanol, heavy paraffin oil, soft white paraffin, cetostearyl alcohol, and macrogol cetostearyl ether; and
an active agent.

2. The pharmaceutical composition of claim 1, wherein the pharmaceutically acceptable carrier comprises between 46.5 and 49.5 weight percent water.

3. The pharmaceutical composition of claim 1, wherein the pharmaceutically acceptable carrier comprises between 4.2 and 5.8 weight percent propylene glycol.

4. The pharmaceutical composition of claim 1, wherein the pharmaceutically acceptable carrier comprises between 0.4 and 0.8 weight percent phenoxyethanol.

5. The pharmaceutical composition of claim 1, wherein the pharmaceutically acceptable carrier comprises between 9.2 and 10.8 weight percent heavy paraffin oil.

6. The pharmaceutical composition of claim 1, wherein the pharmaceutically acceptable carrier comprises between 9.2 and 10.8 weight percent soft white paraffin.

7. The pharmaceutical composition of claim 1, wherein the pharmaceutically acceptable carrier comprises between 14.2 and 15.6 weight percent cetostearyl alcohol.

8. The pharmaceutical composition of claim 1, wherein the pharmaceutically acceptable carrier comprises between 0.4 and 1.0 weight percent macrogol cetostearyl ether.

9. The pharmaceutical composition of claim 1, wherein said active agent comprises an immunological agent such as a vaccine.

10. The pharmaceutical composition of claim 1, wherein said active agent comprises a recombinant protein.

11. The pharmaceutical composition of claim 1, wherein said active agent comprises a recombinant peptide.

12. The pharmaceutical composition of claim 1, wherein said active agent comprises an adjuvant.

13. The pharmaceutical composition of claim 1, wherein said active agent comprises an opioid.

14. The pharmaceutical composition of claim 1, wherein said active agent comprises a non-opioid analgesic.

15. The pharmaceutical composition of claim 1, wherein said active agent comprises a non-steroidal, anti-inflammatory drug.

16. The pharmaceutical composition of claim 1, wherein said active agent comprises a hormone.

17. The pharmaceutical composition of claim 1, wherein said active agent comprises nicotine.

18. The pharmaceutical composition of claim 1, wherein said active agent comprises a nicotine-related substance.

19. A method for transdermal delivery of an active agent, comprising topically applying to a subject in need thereof a pharmaceutical composition according to claim 1.
